(19) United States
(12) Patent Application Publication
(10) Pub. No.: US 2016/0147794 A1
(43) Pub. Date: May 26, 2016

(54) ULTRASONIC DIAGNOSTIC APPARATUS, MEDICAL IMAGE PROCESSING APPARATUS, AND MEDICAL IMAGE PROCESSING METHOD

(71) Applicants: Kabushiki Kaisha Toshiba, Minato-ku (JP); Toshiba Medical Systems Corporation, Otawara-shi (JP)

(72) Inventors: Yasuhiko ABE, Otawara (JP); Tetsuya Kawagishi, Kawasaki (JP)

(73) Assignees: Kabushiki Kaisha Toshiba, Minato-ku (JP); Toshiba Medical Systems Corporation, Otawara-shi (JP)

(21) Appl. No.: 14/945,786

(22) Filed: Nov. 19, 2015

(30) Foreign Application Priority Data
Nov. 25, 2014 (JP) 2014-238275

(51) Int. Cl. G06F 17/30; A61B 8/08; G06T 7/00; A61B 5/055; A61B 6/03; A61B 8/00; A61B 8/14

(52) U.S. Cl. CPC G06F 17/30259; A61B 8/467; A61B 8/52; A61B 8/14; A61B 8/486; A61B 8/488; A61B 5/055; A61B 6/032; G06T 7/004

(57) ABSTRACT

According to one embodiment, an ultrasonic diagnostic apparatus includes a plurality of types of databases for detecting a contour position of a predetermined region of an object, input interface circuitry, and detection circuitry. The input interface circuitry designates a database, of the plurality of databases, which is desired by a user. The detection circuitry detects a contour position of the predetermined region on an ultrasonic image as an input image by using the designated database.

### CROSS-REFERENCE TO RELATED APPLICATIONS

This application is based upon and claims the benefit of priority from prior Japanese Patent Application No. 2014-238275, filed Nov. 25, 2014, the entire contents of which are incorporated herein by reference.

### FIELD

Embodiments described herein relate generally to an ultrasonic diagnostic apparatus, medical image processing apparatus, and medical image processing method which use a knowledge-based dictionary for automatically detecting the contour of an examination region such as a cardiac lumen.

### BACKGROUND

Ultrasonic image diagnosis has been widely used, in which an object is irradiated with ultrasonic waves, and a scanned region is imaged by using the reflected waves, thereby allowing an examiner such as a doctor to perform proper diagnosis. For example, it is often the case in which the contour (or the boundary position) of a cardiac lumen is acquired by using a diagnostic image used for ultrasonic image diagnosis, such as a B-mode image, an MPR image obtained from volume data, an M-mode image, or a Doppler image, and for example, a predetermined measurement value such as the volume of the cardiac lumen is acquired by using the acquired contour.

Recently, there has been known ACT (Automated Contour Trace) which uses a knowledge-based dictionary (recognition type database or shape dictionary) to acquire the above contour. Assume that in this specification, a knowledge-based dictionary indicates a database which is constructed through the learning process of using images for the organization of a database as inputs and letting a teacher give contours (boundary positions) as answers. ACT techniques include a fully automatic means which requires no auxiliary operation by the user as an examiner and a means for making the user designate several predetermined positions on a living structure and detecting a proper contour based on the designated points. When using any of these means, a knowledge-based dictionary is created or updated by using contours taught by an examiner as learning sources.

Ultrasonic images (echo images) obtained by an ultrasonic diagnostic apparatus have low resolution and contain many acoustic artifacts as compared with magnetic resonance imaging images (MRI images) obtained by magnetic resonance imaging apparatus and X-ray computed tomographic images (CT images) obtained by an X-ray computed tomography apparatus. For this reason, the examiner recognizes different contours from even tomographic images of the same anatomical region (e.g., tomographic images of a cardiac lumen) mainly because of resolution differences. FIG. 6 shows an example of contour definitions (echo definitions) using an echo image I1. Referring to FIG. 6, the thick lines indicate an endocardial position P1 and an epiocardial position P2 which are recognized on the left ventricular myocardium. FIG. 7 shows an example of contour definitions (MRI definitions) using an MRI image 12. Referring to FIG. 7, the thick lines indicate an endocardial position P3 and an epiocardial position P4 which are recognized on the left ventricular myocardium. The echo image I1 has low sharpness on a boundary region. That is, blur occurs on the boundary region. For this reason, the endocardial position P1 based on the echo definition tends to be recognized as being located inside compared with the endocardial position P3 based on the MRI definition or the endocardial position based on the contour definition using a high-resolution CT image. Even with the same contour definition, variation (variance) often occur in the recognition of contours depending on experiences of examiners.

If a learning source includes a contour difference based on image resolutions and variation in contour recognized by each examiner when creating or updating a knowledge-based dictionary, the reliability and objectivity with respect to contours detected by ACT deteriorate.

### BRIEF DESCRIPTION OF THE DRAWINGS

FIG. 1 is a block diagram showing the arrangement of an ultrasonic diagnostic apparatus according to an embodiment;

FIG. 2 is a view showing a position based on an echo definition and a position based on an MRI definition with respect to an echo image of an apical four chamber view;

FIG. 3 is a flowchart showing the operation of the ultrasonic diagnostic apparatus shown in FIG. 1;

FIG. 4 is a view showing an M-mode image with respect to an echo image of a parasternal long-axis image;

FIG. 5 is a view showing a CW Doppler waveform with respect to a Doppler image of an apical three chamber view;

FIG. 6 is a view showing echo definitions using an echo image; and

FIG. 7 is a view showing MRI definitions using an MRI image.

### DETAILED DESCRIPTION

In general, according to one embodiment, an ultrasonic diagnostic apparatus includes a plurality of types of databases for detecting a contour position of a predetermined region of an object, input interface circuitry, and detection circuitry. The input interface circuitry designates a database, of the plurality of databases, which is desired by a user. The detection circuitry detects a contour position of the predetermined region on an ultrasonic image as an input image by using the designated database.

This embodiment will be described below with reference to the accompanying drawings. Note that the same reference numerals in the following description denote constituent elements having almost the same arrangements, and a repetitive description will be made only when required.

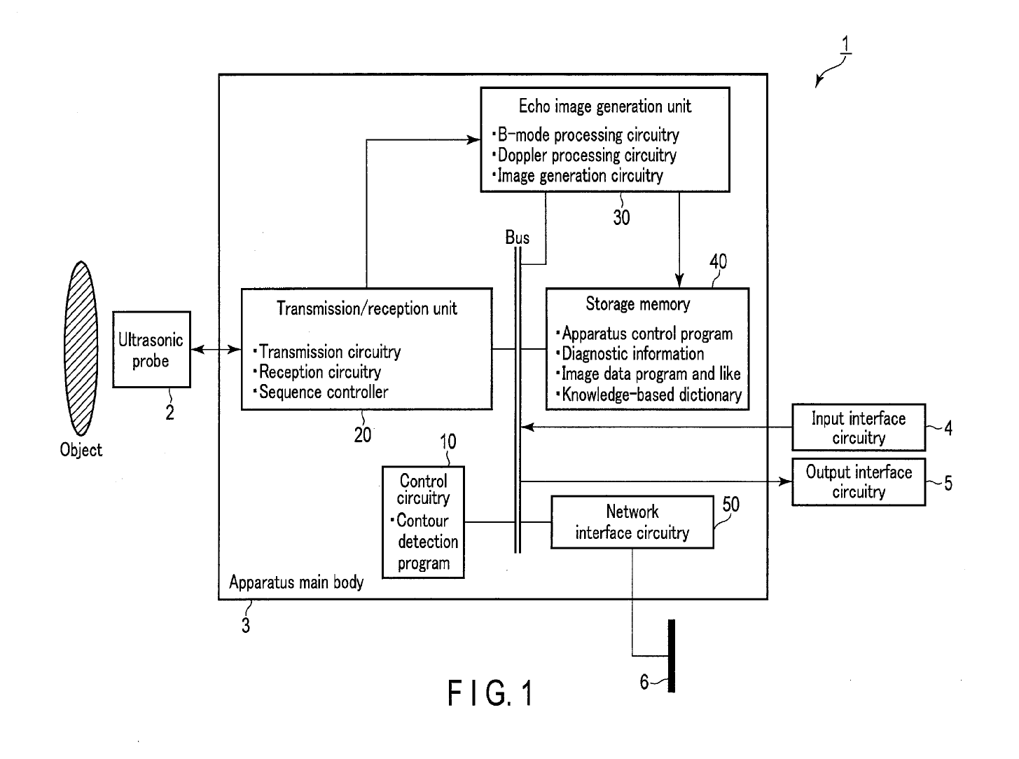

FIG. 1 is a block diagram showing the arrangement of an ultrasonic diagnostic apparatus 1 according to an embodiment. The ultrasonic diagnostic apparatus 1 includes an ultrasonic probe 2, an apparatus main body 3, input interface circuitry 4, and output interface circuitry 5. A network 6 capable of communicating with the outside may be connected to the ultrasonic diagnostic apparatus 1 via network interface circuitry 50 of the apparatus main body 3.

The ultrasonic probe 2 includes a plurality of piezoelectric transducers, a matching layer, and a backing member provided on the back side of the plurality of piezoelectric transducers. The plurality of piezoelectric transducers are acoustoelectric reversible conversion elements such as piezoelectric ceramic elements. The plurality of piezoelectric transducers are juxtaposed and provided on the distal end of the ultrasonic probe 2. The piezoelectric transducers generate ultrasonic waves in response to driving pulses transmitted and supplied from the transmission circuitry of a transmission/reception unit 20.

When an object is irradiated with ultrasonic waves via the ultrasonic probe 2, the applied ultrasonic waves are reflected by a discontinuity surface of acoustic impedance of internal body tissue in the object. The piezoelectric transducers receive the reflected ultrasonic waves and generate echo signals. Upon receiving an echo signal, reception circuitry in the transmission/reception unit 20 transmits a reception signal based on the received echo signal to an echo image generation unit 30.

The amplitude of an echo signal depends on an acoustic impedance difference on a discontinuity surface as a boundary by which ultrasonic waves are reflected. As a transmission piezoelectric transducer for generating ultrasonic waves and a reception piezoelectric transducer for generating echo signals, the same piezoelectric transducer may be selectively used by deciding a transmission timing and a reception timing when applying discrete ultrasonic waves (pulse waves). Alternatively, for example, when applying continuous ultrasonic waves (continuous waves), transmission piezoelectric transducers and reception piezoelectric transducers may be separately mounted on the ultrasonic probe 2. The frequency of an echo signal produced when a transmission ultrasonic wave is reflected by a moving blood flow, the surface of the moving cardiac wall, or the like (to be referred to as a moving body hereinafter) is subjected to a frequency shift depending on the velocity component of the moving body in the ultrasonic transmission direction due to the Doppler effect.

The apparatus main body 3 includes control circuitry 10, the transmission/reception unit 20, the echo image generation unit 30, storage circuitry 40, and the network interface circuitry 50.

The control circuitry 10 is a processor which mainly controls the overall operation of the ultrasonic diagnostic apparatus 1. In addition, the control circuitry 10 is a processor which reads out a contour detection program from the storage circuitry 40 and executes the readout contour detection program. More specifically, the control circuitry 10 receives an instruction to select a desired type of knowledge-based dictionary, which is input by the user. The control circuitry 10 reads out the desired type of knowledge-based dictionary of a plurality of types of knowledge-based dictionaries stored in the storage circuitry 40 in response to the selection instruction. The control circuitry 10 executes ACT by using the readout knowledge-based dictionary.

The transmission/reception unit 20 includes, for example, transmission circuitry, reception circuitry, and a sequence controller.

As described above, the transmission circuitry transmits driving pulses to a plurality of piezoelectric transducers of the ultrasonic probe 2.

As described above, the reception circuitry generates a reception signal based on an echo signal generated by each piezoelectric transducer. The reception circuitry transmits the generated reception signals to the echo image generation unit 30.

The sequence controller controls the transmission sequence of driving pulses for the generation of ultrasonic waves with a high time resolution on the order of several ms.

The echo image generation unit 30 includes, for example, B-mode processing circuitry, Doppler processing circuitry, and image generation circuitry.

The B-mode processing circuitry includes envelope detection circuitry and logarithmic conversion circuitry. The envelope circuitry is a processor which executes envelope detection with respect to a reception signal output from the reception circuitry. The envelope detection circuitry outputs the envelope-detected signal to the logarithmic conversion circuitry. The logarithmic conversion circuitry is a processor which relatively enhances a weak signal by logarithmically converting the envelope-detected signal. The B-mode processing circuitry generates a signal value for each depth on each scanning line or in each ultrasonic transmission/reception based on the signal enhanced by the logarithmic conversion circuitry. Note that the B-mode processing circuitry may generate volume data in place of the signal value for each depth on each scanning line or in each ultrasonic transmission/reception. Data generated by the B-mode processing circuitry will be collectively referred to as B-mode data hereinafter.

Note that the B-mode processing circuitry may be a processor which implements a B-mode processing function of generating B-mode data by reading out an operation program from the storage circuitry 40 and executing the readout operation program.

The Doppler processing circuitry includes a mixer, an LPF (Low Pass Filter), and velocity/variance/power computation circuitry. The mixer multiplies a reception signal output from the reception circuitry by a reference signal having a frequency f₀ equal to the transmission frequency. This multiplication obtains a signal having a component with a Doppler shift frequency f_{d} and a signal having a frequency component of (2f₀+f_{d}). The LPF removes a signal of a high-frequency component (2f₀+f_{d}) from a signal having two types of frequency components from the mixer. The LPF generates a Doppler signal having the component with the Doppler shift frequency f_{d} by removing the signal of the high-frequency component (2f₀+f_{d}). A Doppler signal is, for example, a Doppler component based on a blood flow, tissue, and contrast medium.

Note that the Doppler processing circuitry may use a quadrature detection scheme to generate Doppler signals. In this case, the Doppler processing circuitry performs quadrature detection to convert a reception signal into an IQ signal. The Doppler processing circuitry generates a Doppler signal having the Doppler shift frequency f_{d} by performing complex Fourier transform of the IQ signal.

The velocity/variance/power computation circuitry includes an MTI (Moving Target Indicator) filter, an LPF filter, and autocorrelation computation circuitry. The MTI filter removes a Doppler component (clutter component) caused by the respiratory movement or pulsatory movement of an organ or the like from a generated Doppler signal. The MTI filter is used to extract a Doppler component concerning a blood flow from a Doppler signal. The LPF is used to extract a Doppler component concerning the movement of the tissue from a Doppler signal. Note that the velocity/variance/power computation circuitry may include cross-correlation computation circuitry instead of the autocorrelation computation circuitry.

Note that the Doppler processing circuitry may be a processor which implements a Doppler processing function of generating Doppler signals by reading out an operation program from the storage circuitry 40 and executing the readout operation program.

The image generation circuitry includes a DSC (Digital Scan Converter) and an image memory. The image generation circuitry executes coordinate conversion processing (resampling) for the DSC. Coordinate conversion processing is to convert, for example, a scanning line signal string for ultrasonic scanning, which is formed from B-mode data, Doppler data, and propagation time data, into a scanning line signal string in a general video format typified by a TV format. The image generation circuitry generates an echo image as a display image by executing coordinate conversion processing. More specifically, the image generation circuitry generates a B-mode image based on B-mode data. The image generation circuitry generates a Doppler image such as an average velocity image, a variance image, or a power image based on Doppler data. In addition, the image generation circuitry may generate a superimposed image by combining an echo signal with presentation information of a contour detected by a contour detection program (to be described later), character information (annotation) of various types of parameters, scale marks, and the like.

Note that the image generation circuitry may be a processor which implements a coordinate conversion processing function by reading out an operation program from the storage circuitry 40 and executing the readout operation program.

The storage circuitry 40 stores control programs for the ultrasonic diagnostic apparatus 1, a diagnostic protocol, various types of data groups such as transmission/reception conditions, diagnostic information (patient IDs, findings by doctors, and the like), reception signals generated by the reception circuitry, B-mode data generated by the B-mode processing circuitry, Doppler data generated by the Doppler processing circuitry, and a plurality of types of knowledge-based dictionaries.

The storage circuitry 40 includes a memory (not shown) and stores data (image data) corresponding to generated echo signals (B-mode images, average velocity images, variance images, power images, and the like). Image data stored in the memory is read out in accordance with an instruction from the operator via the input interface circuitry 4. This memory stores, for example, echo images corresponding to a plurality of frames immediately before freezing. Continuously displaying (cine displaying) the images stored in this cine memory can output echo images to the output interface circuitry 5.

The network interface circuitry 50 is connected to the network 6. Data such as echo images, analysis results, and the like which are obtained by the apparatus main body 3 can be transferred to other apparatuses via the network interface circuitry 50 and the network 6. In addition, the network interface circuitry 50 can also download, via the network 6, medical images concerning objects acquired by other medical image diagnostic apparatuses.

The input interface circuitry 4 is implemented by a trackball, switch buttons, a mouse, a keyboard, a touch pad for an input operation for the execution of an input operation by a touch on an operation screen, a touch panel display integrating a display screen and a touch pad, and the like, which are used to, for example, set an ROI (Region Of Interest). The input interface circuitry 4 is connected to the control circuitry 10 to convert input operations for various types of instructions, commands, information, selections, settings, and the like received from the user into electrical signals and output them to the control circuitry 10. The user, in particular, selects a proper type of knowledge-based dictionary from a plurality of types of knowledge-based dictionaries via the input interface circuitry 4.

Note that in this specification, the input interface circuitry 4 is not limited to the one including physical operating parts such as a mouse and a keyboard. For example, the input interface circuitry 4 may also include, as an example, an electrical signal processing circuitry which receives electrical signals corresponding to input operations from an external input device provided separately from the apparatus and outputs the electrical signals to the control circuitry 10.

The output interface circuitry 5 includes, for example, a monitor such as a CRT or liquid crystal display. The output interface circuitry 5 displays echo images generated by the image generation circuitry and echo images (superimposed images) including the presentation information of contours detected by ACT.

### (Type of Knowledge-Based Dictionary)

A plurality of types of knowledge-based dictionaries (axes a, b, and c) of the ultrasonic diagnostic apparatus 1 will be described.

### [Axis a (First Type)]: Differences in “Definition” Position Between Modalities

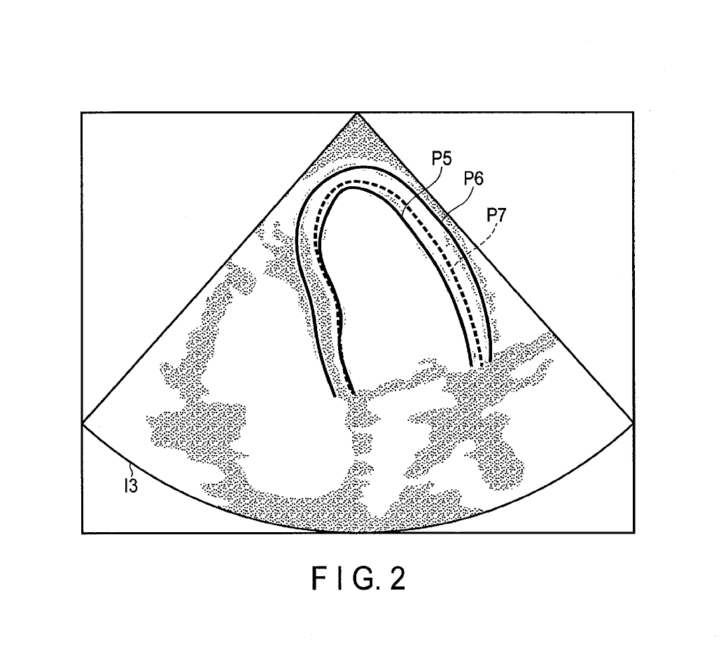

The ultrasonic diagnostic apparatus 1 gives the user a degree of freedom to select any of the following different definitions: “a knowledge-based dictionary obtained from endocardial boundary positions (MRI definitions) estimated by a diagnostic professional using MRI images which make it difficult to mistake trabeculae carneae and tendinous cords for ecdocadium” and “a knowledge-based dictionary obtained from an endocardial boundary position definitions (echo definitions) estimated by a standard diagnostic professional using echo images”. FIG. 2 shows an example of echo definition positions and an MRI definition position on an echo image I3 of an apical four chamber view. Referring to FIG. 2, the solid lines indicate an endocardial position P5 and an epiocardial position P6 on the left ventricular myocardium based on echo definitions, and the broken line indicates an endocardial position P7 on the left ventricular myocardium based on an MRI definition. The endocardial position P7 based on the MRI definition is located more outside the cardiac chamber side on the cardiac apex and the lateral wall side than the endocardial position P5 based on the echo definition. As described above, it is obvious that the two definition positions are not the same, and the differences vary depending on the position of a structure.

### [Axis b (Second Type)]: Differences in “Recognition” Position Between Examiners

### (B-1)

The ultrasonic diagnostic apparatus 1 gives the user a degree of freedom to select any of the following dictionaries: “a knowledge-based dictionary based on positions taught by a teacher XX who is an expert (ultrasonic diagnostic expert)” and “a knowledge-based dictionary based on average positions taught by many ultrasonic diagnostic professionals”. That is, the ultrasonic diagnostic apparatus 1 gives the user a degree of freedom to select a concept for reducing variation (variance) in recognition of a knowledge-based dictionary. This is because, even if there are differences in recognition position depending on teachers, when creating a dictionary to bring boundary positions near to actual boundary positions (virtual true positions), there are available a means based on teaching of positions by an expert and a means based on teaching of positions by many professionals. Note that a knowledge-based dictionary based on the latter concept may use average positions by a plurality of experts.

CT images relatively facilitate discrimination between myocardial tissues and blood based on CT values. In contrast, the luminance signals of echo images have no distinct physical unit. For this reason, it is difficult to clearly discriminate between the boundary between the myocardium and the inside of the cardiac chamber (blood) based on an echo image. Therefore, the recognition of boundary positions includes human's determination. For this reason, contour positions in ultrasonic diagnosis are estimated as virtual boundary positions between the myocardium and the cardiac chamber.

In such conditions, widespread studies have generally been conducted on the above two concepts for reducing the influence of variation in individual recognition. According to the former concept, using the presence of the recognition bias of each expert as an underhanded way will reduce variation by properly selecting an expert having an average recognition bias. According to the latter concept, averaging the degrees of recognition biases of the individuals will reduce variation.

### (B-2)

As a modification, the ultrasonic diagnostic apparatus 1 may have a learning function of updating dictionary data by learning a knowledge-based dictionary like a learning type kanji conversion dictionary widely used in PCs, cellular phones, and the like. For example, the control circuitry 10 implements a learning function by reading out a learning program from the storage circuitry 40 and executing the readout learning program. The ultrasonic diagnostic apparatus 1 gives the user a degree of freedom to select knowledge-based dictionaries, while grasping the attributes of each creation source for knowledge-based dictionaries individually created by different facilities or knowledge-based dictionaries individually created by different examiners, like “hospital facility A, B, and or C” and “examiner x, y, or z”. This differs from (b-1). That is, the ultrasonic diagnostic apparatus 1 learns a rule for each facility or the preference and rule of each examiner as an individual knowledge-based dictionary and allows the user to select a knowledge-based dictionary updated based on the learning. In this case, the user may select, as a dictionary type, a knowledge-based dictionary based on (b-1) or a knowledge-based dictionary based on (b-2).

In addition, the apparatus may create a plurality of knowledge-based dictionaries by learning from teachers YY and ZZ as experts indicated by (b-1) using the arrangement of (b-2) and allows the user to select one of them as a dictionary type.

This ensures objectivity by specifying a facility or examiner as the creation source of a knowledge-based dictionary even if the contents of the knowledge-based dictionary provided by the business professional who creates knowledge-based dictionaries are dynamically changed.

When creating a knowledge-based dictionary, the business professional can make a predetermined facility or examiner automatically learn contour positions until the number of input applications of contour information by the user reaches a predetermined number in routine examinations on the apparatus. This reduces the time and effort to create a knowledge-based dictionary.

In this case, when the user performs contour setting by a manual means or an automatic means such as ACT and performs a measuring operation based on the set contours, contour information is automatically regarded as input applications. Alternatively, a dedicated user interface is provided, and the user explicitly performs input application while regarding a currently set state as a proper setting. In accordance with this input application, a knowledge-based dictionary is learnt, and dictionary data is updated.

### [Axis c (Third Type)]: Differences Between “Resolutions” of Reference Images

The ultrasonic diagnostic apparatus 1 gives the user a degree of freedom to select, as images (reference images) used for the creation of a knowledge-based dictionary, between different images including “an echo image group (equivalent echo images) having a resolution equivalent to an echo image group used for contour detection” and “an MRI image group or CT image group (different modality images) higher in resolution than an echo image group used for contour detection”. The axes a and b are set on the assumption that images used for the creation of a dictionary are echo images of the same type as that of images actually used for contour detection. This is because, if the type of images (reference images) used for the creation of a knowledge-based dictionary is the same as that of input images for contour detection which are collated with the knowledge-based dictionary, it is generally easy to improve the accuracy of collation since they have similar luminance information patterns. In this case, it is preferable to form a knowledge-based dictionary by using both the vector information of shape spaces obtained from contours and luminance information of the reference images.

When, however, creating a knowledge-based dictionary based on definitions different from echo definitions as indicated by the axis a, it is natural to determine boundary positions by using, for example, MRI images, and create a knowledge-based dictionary by using MRI images. This is because, teaching contour positions based on different definitions such as the axis a by using echo images will force the teacher to have sophisticated experience and perform sophisticated determination for a specific boundary position when, for example, using MRI.

In this case, a knowledge-based dictionary is formed by using only the vector information of shape spaces obtained from contours. At the time of collation, a contour position Cb based on luminance information is estimated from an input echo image, and is compared/collated with a contour position Cd based on the knowledge-based dictionary to detect a final contour position Cb′ by deforming the contour position Cb as a position nearest to the contour position Cd under restrictions based on a predetermined criterion. As the predetermined criterion, for example, contour shape energy minimization method or the like known as a Snakes algorithm is used.

Note that as images in the latter case, an echo image group (high-frequency echo images) with a higher resolution, which is obtained by using a probe with a high center frequency, may be used in addition to different modality images. For example, it is preferable to use, as echo images used for contour detection, images obtained by a probe in a band with a center frequency of 3 MHz, which is mainly used for adults and to use, as images for the creation of a knowledge-based dictionary, images obtained by a probe in a band with a center frequency of 5 MHz, which is mainly used for children.

In this case, since echo images are used for the creation of a knowledge-based dictionary and for contour detection, it is possible to reduce the restrictions imposed on a teacher at the time of the creation of a knowledge-based dictionary and add luminance information to the knowledge-based dictionary. In addition, using ultrasonic images with a higher resolution can reduce recognition errors concerning boundary positions of lumens which are caused by blur.

With these operations, the user can obtain different types of contour positions depending on ACT to be used by selecting a knowledge-based dictionary depending on the intended use. In a specific use, while an echo definition position is used for the measurement of a lumen volume, an MRI definition position can be selected as needed, thereby reducing the degree of underestimation of the lumen volume in MRI. In addition, it is possible to select a specific concept to reduce differences in recognition position between examiners at the time of the creation of a knowledge-based dictionary.

Alternatively, it is also possible to perform selection with respect to differences in recognition position between examiners so as to follow an authoritative site or identifiable expert examiner. Aside from this, it is also possible to explicitly select what type of modality images are used for the creation of a knowledge-based dictionary.

### (First Modification)

The above three types of axes are independent of each other. It is therefore possible to obtain a new type of axis by combining two different types of axes from the axes a, b, and c. Since the type of axis obtained by combining the above axes includes the effect and function associated with the individual types of axes described above in a composite manner, each type of axis produces a unique meaning.

The user can select a knowledge-based dictionary optimal for the intended use in consideration of these individual meanings. In this case, it is preferable to display supplementary explanations about features (the above unique meanings) provided by the respective knowledge-based dictionaries existing as choices when selecting a knowledge-based dictionary, in order to allow the user to easily grasp the features.

Note that contour recognition using a knowledge-based dictionary may be applied to not only B-mode 2D slice images but also slice images such as MPR images obtained from volume data as input images. A target organ (predetermined region) is limited to the heart and may be an arterial vessel such as a carotid artery. The contours of the detected ebdocadium and epicardium may be used as the initial contours from which tracking starts, which are regarded as a region of interest in the myocardium when obtaining a cardiac function index value such as a strain by the ST (Speckle-Tracking) method.

### (Operation Example)

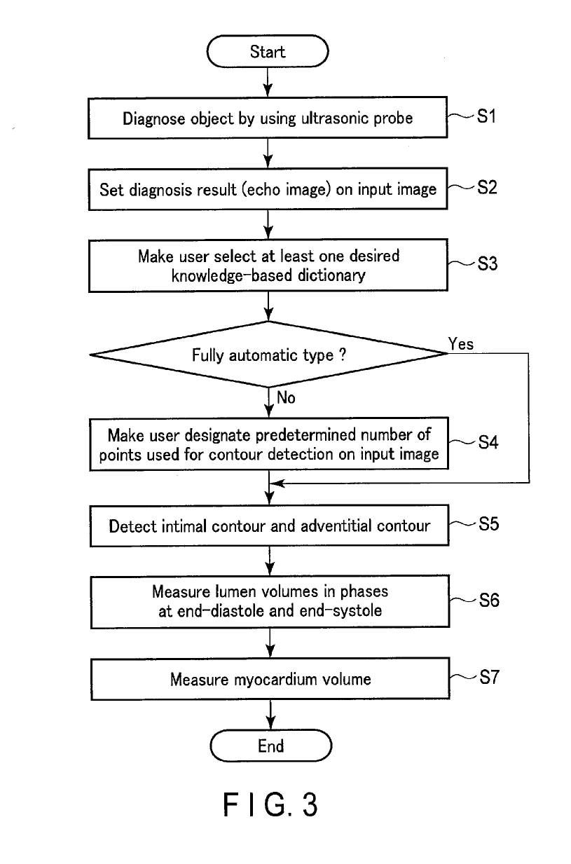

FIG. 3 is a flowchart showing an operation example of the ultrasonic diagnostic apparatus 1 according to the embodiment. Assume that a 2D slice image (B-mode image) is handled here. An operation example will be described below along with the respective steps shown in FIG. 3.

### (Step S1)

The user performs diagnosis by using an ultrasonic image obtained by transmitting and receiving ultrasonic waves to and from an object while holding the ultrasonic probe 2 in his/her hand. This diagnosis result is obtained as an echo image (a B-mode image as a moving image over a predetermined period) generated by the echo image generation unit 30.

### (Step S2)

The user inputs an instruction to set, via the input interface circuitry 4, as an input image, an echo image in a predetermined phase, of echo images generated by the echo image generation unit 30 over a predetermined period. The control circuitry 10 sets an echo image generated by the echo image generation unit 30 as an input image by executing a predetermined program stored in the storage circuitry 40 in response to the instruction.

### (Step S3)

The user inputs an instruction to select at least one desired type of knowledge-based dictionary via the input interface circuitry 4. Note that this desired type is preferably registered in advance as a preset setting in a predetermined program which has been executed. This makes the user select a type as a default setting without explicitly inputting the type of the knowledge-based dictionary. The control circuitry 10 sets the selected knowledge-based dictionary as a knowledge-based dictionary used for contour detection by executing a predetermined program stored in the storage circuitry 40.

### (Step S4)

The user inputs an instruction to designate a predetermined number of points used for contour detection via the input interface circuitry 4. The control circuitry 10 sets the designated predetermined number of points by executing a predetermined program stored in the storage circuitry 40 in response to the instruction. Note that this step is omitted when fully automatic ACT is used.

### (Step S5)

The control circuitry 10 detects an endocardial contour and an epicardial contour by executing a predetermined program stored in the storage circuitry 40 based on the at least one desired knowledge-based dictionary set in step S3 and the predetermined number of points set in step S4.

### (Step S6)

Based on the endocardial contour and the epicardial contour detected in step S5, the control circuitry 10 measures lumen volumes (EDV and ESV) in predetermined phases, for example, a phase at an end-diastole (ED) and a phase at an end-systole (ES) by executing a predetermined program stored in the storage circuitry 40.

### (Step S7)

Based on the lumen volumes in the phases at the end-diastole and the end-systole measured in step S6, the control circuitry 10 measures a myocardial volume by executing a predetermined program stored in the storage circuitry 40.

### (Second Modification)

In ultrasonic image diagnosis, M-mode images, Doppler images, and the like are used in addition to B-mode images, and predetermined measurement using them is performed.

In the ultrasonic diagnostic apparatus 1 according to the embodiment, ACT based on the plurality of types of knowledge-based dictionaries may be applied to M-mode images and Doppler images in addition to 2D images (B-mode images).

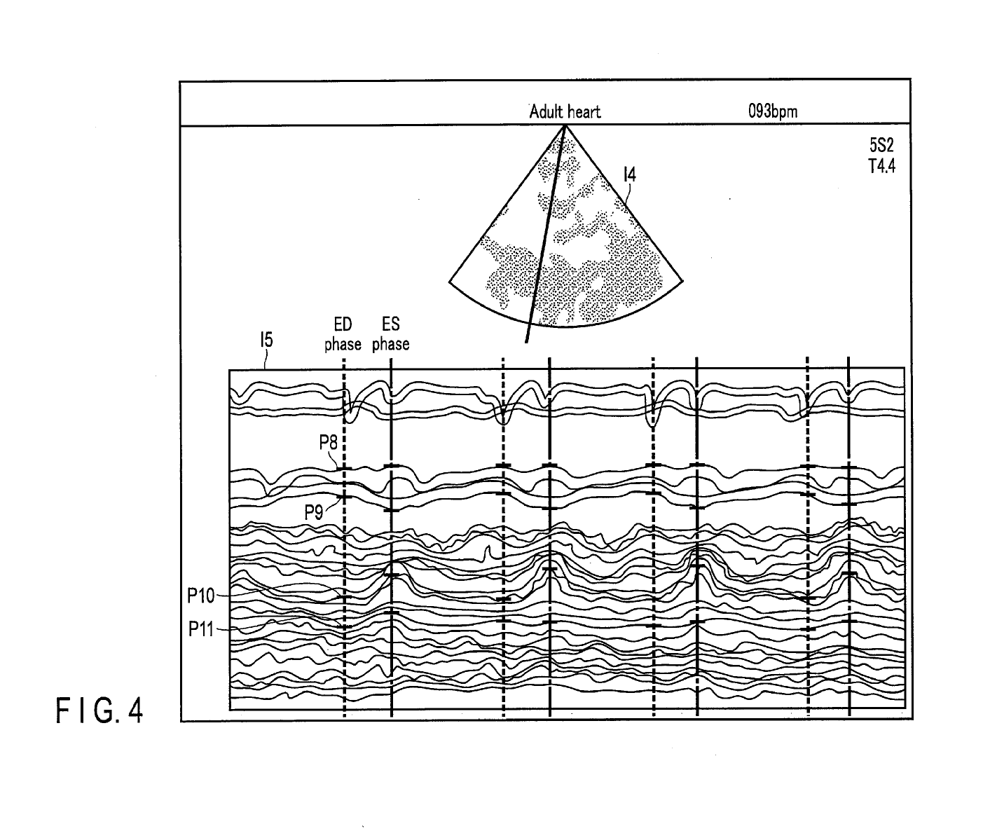

FIG. 4 is a view showing an example of an M-mode image corresponding to an echo image of a parasternal long-axis image. An M-mode image I5 in FIG. 4 represents temporal displacements of tissues on the thick line on an echo image 14. The broken lines and the one-dot dashed lines on the M-mode image I5 respectively represent ED phases and ES phases. In addition, P8, P9, P10, and P11 on the M-mode image I5 respectively represent an epiocardial position on the anteroseptal side, an endocardial position on the anteroseptal side, an endocardial position on the posterior wall side, and an epiocardial position on the posterior wall side. It is difficult to detect a myocardium boundary position in an M-mode image as a simple luminance information edge. In FIG. 4, many boundary position candidates are recognized on the posterior wall side, in particular. In order to automatically obtain a predetermined myocardium boundary position from such an image, it is preferable to perform automatic detection based on a knowledge-based dictionary.

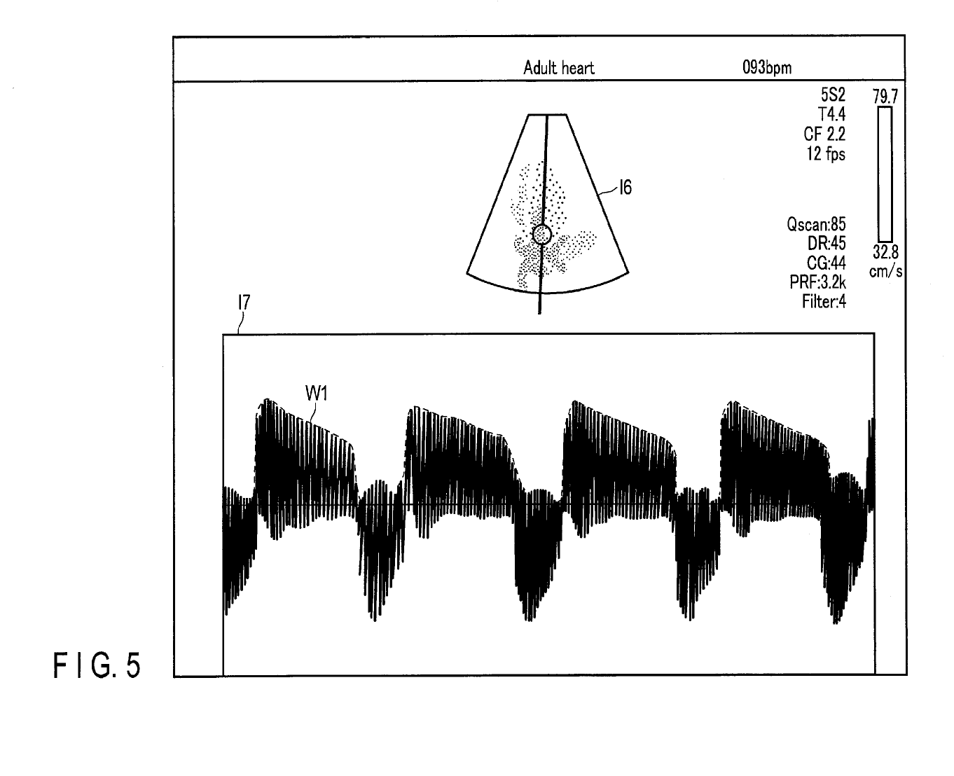

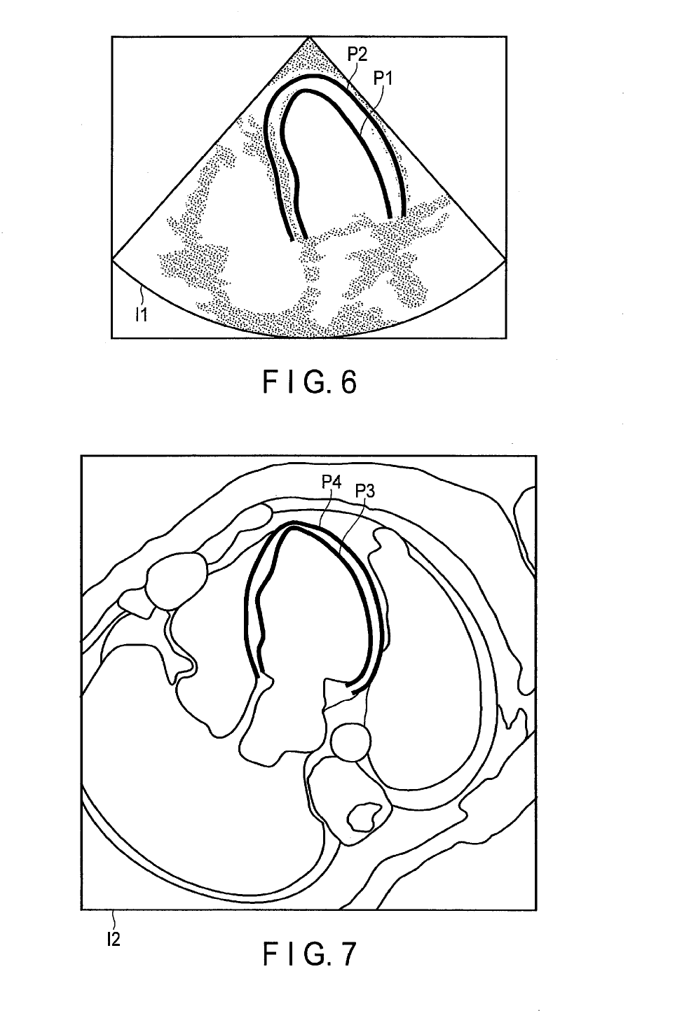

FIG. 5 is a view showing an example of a CW Doppler waveform corresponding to a Doppler image of an apical three chamber view. A CW Doppler waveform W1 on a waveform image 17 in FIG. 5 represents the velocity of the region surrounded by the circle on a Doppler image 16. The broken line superimposed on the CW Doppler waveform W1 is a trace line indicating the envelope positions of aortic valve regurgitation at end-diastoles. Referring to FIG. 5, a left ventricular blood outflow, which is a normal blood flow, and mitral regurgitation are mixed at each end-systole. That is, this is not a simple state that allows detection of only a predetermined envelope. In addition, the recognition position of the boundary position of an envelope varies depending on Doppler gain settings. In order to automatically obtain a predetermined boundary position of the envelope from such an image, it is preferable to perform automatic detection based on a knowledge-based dictionary.

When automatically detecting a boundary position from such an image based on a knowledge-based dictionary, the user can select a predetermined type of knowledge-based dictionary from a plurality of types of knowledge-based dictionaries in accordance with the intended use.

An axis b (b-1 and b-2) can be applied as another type of axis to both an M-mode image and a Doppler image.

In addition, with regard to M-mode images, the axis c can be applied to a case in which an echo image group (high-frequency echo images) with a higher resolution obtained by using a probe with a high center frequency is used.

### (Effects)

The ultrasonic diagnostic apparatus 1 according to the embodiment can obtain the following effects.

The ultrasonic diagnostic apparatus 1 according to the embodiment has a plurality of types of knowledge-based dictionaries respectively based on differences in “definition” position between modalities (first type), differences in “recognition” position between examiners (second type), and differences between the “resolutions” of images used for the creation of a knowledge-based dictionary (third type).

With this arrangement, when the user selects the first type of knowledge-based dictionary, it is possible to reduce the degree of underestimation of a lumen volume in MRI by selecting MRI definitions as needed while basically using echo definitions.

When the user selects the second type of knowledge-based dictionary, it is possible to provide the user with a degree of freedom to select a specific concept to reduce differences in recognition position between examiners at the time of the creation of a knowledge-based dictionary with respect to each position definition. It is also possible to perform selection with respect to differences in recognition position between examiners so as to follow an authoritative site and/or identifiable expert examiner.

When the user selects the third type of knowledge-based dictionary, it is possible to provide the user with a degree of freedom to select between an echo image group having a resolution equivalent to echo images used as input images and different modality images having a higher resolution.

In addition, the above three types of knowledge-based dictionaries are not exhaustive; it is possible to create a new type of knowledge-based dictionary while implementing the effects of the respective types in a composite manner by combining at least two of the three types of knowledge-based dictionaries.

The word “processor” used in the above description means circuitry such as a CPU (Central Processing Unit), a GPU (Graphics Processing Unit), an ASIC (Application Specific Integrated Circuit), a programmable logic device (e.g., an SPLD (Simple Programmable Logic Device), a CPLD (Complex Programmable Logic Device), or an FPGA (Field Programmable Gate Array)), or the like. The processor implements functions by reading out programs stored in the storage circuit 40 and executing them. Note that it is possible to directly incorporate programs in the circuitry of the processor instead of storing them in the storage circuitry 40. In this case, the processor implements functions by reading out programs incorporated in the circuitry and executing them. Note that each processor in each embodiment described above may be formed as one processor by combining a plurality of independent circuits to implement functions as well as being formed as single circuitry for each processor. In addition, a plurality of constituent elements in FIG. 1 may be integrated into one processor to implement its function.

The above embodiment has exemplified the case in which the ultrasonic diagnostic apparatus executes automatic detection of a contour by using a knowledge-based dictionary. However, the embodiment is not limited to this. For example, a medical image processing apparatus having an automatic contour detection function using a knowledge-based dictionary may execute the above operation by transferring cardiac image data obtained by the ultrasonic diagnostic apparatus to a computer such as a PC or workstation. In this case, the medical image processing apparatus may use, as input images, images obtained by not only the ultrasonic diagnostic apparatus but also, for example, an X-ray computed tomography apparatus and a magnetic resonance imaging apparatus. Alternatively, the above automatic contour detection may be implemented by installing a dedicated medial image processing program for the execution of an automatic contour detection function using knowledge-based dictionaries and activating the program.

Note that the present invention is not limited to the embodiments described above, and constituent elements can be modified and embodied in the execution stage within the spirit and scope of the invention. In addition, various inventions can be formed by proper combinations of a plurality of constituent elements disclosed in the above embodiments. For example, several constituent elements may be omitted from all the constituent elements disclosed in the above embodiments. Furthermore, constituent elements in different embodiments may be properly combined.

1. An ultrasonic diagnostic apparatus comprising:
a plurality of types of databases for detecting a contour position of a predetermined region of an object;
input interface circuitry configured to designate a database, of the plurality of databases, which is desired by a user; and
detection circuitry configured to detect a contour position of the predetermined region on an ultrasonic image as an input image by using the designated database.

2. The apparatus of claim 1, wherein the plurality of types of databases include a definition of a contour position of the predetermined region determined by a standard ultrasonic diagnostic professional and a definition of a contour position of the predetermined region determined based on an image different from an ultrasonic image.

3. The apparatus of claim 1, wherein the plurality of types of databases include a contour position of the predetermined region recognized by a specific ultrasonic diagnostic expert and an average position of a contour position of the predetermined region recognized by a plurality of standard ultrasonic diagnostic professionals.

4. The apparatus of claim 1, wherein the plurality of types of databases include a contour position of the predetermined region obtained from a reference image having a resolution different from that of the input image.

5. The apparatus of claim 4, wherein the reference image comprises a magnetic resonance imaging image, an X-ray computed tomographic image, or an ultrasonic image having a higher spatial frequency than the input image.

6. The apparatus of claim 1, further comprising learning circuitry configured to update at least one of the plurality of types of databases by learning an operation by a teacher,
wherein the plurality of types of databases are constructed for each hospital facility which has performed the learning or for the each teacher.

7. The apparatus of claim 1, wherein the input interface circuitry designates at least two of the plurality of types of databases, and
the detection circuitry detects a contour position of the predetermined region by using the designated at least two databases.

8. The apparatus of claim 1, wherein the input image comprises a slice image, M-mode image, or Doppler waveform image of the predetermined region.

9. A medical image processing apparatus comprising:
a storage unit configured to store an input image including a predetermined region of an object and a plurality of types of databases for detecting a contour position of the predetermined region of the object;
input interface circuitry configured to designate a knowledge-based dictionary including a database, of the plurality of types of databases, which is desired by a user; and
detection circuitry configured to detect a contour position of the predetermined region on a predetermined input image by using the designated knowledge-based dictionary.

10. The apparatus of claim 9, wherein the input image comprises an ultrasonic image, a magnetic resonance imaging image, or an X-ray computed tomographic image.

11. A medical image processing method comprising:
receiving a designation with respect to a knowledge-based dictionary which is created from a plurality of types of elements to detect a contour position of a predetermined region of an object and includes an element, of the plurality of types of elements, which is desired by a user; and
detecting a contour position of the predetermined region on an input image by using the designated knowledge-based dictionary.

12. The method of claim 11, wherein the input image comprises an ultrasonic image, a magnetic resonance imaging image, or an X-ray computed tomographic image.
